(19) United States
(12) Plant Patent Application Publication
(10) Pub. No.: US 2008/0289070 P1
(43) Pub. Date: Nov. 20, 2008

(54) Kiwifruit variety named 'HORT22D'

(76) Inventors: Russell G. Lowe, Te Puke (NZ); Canhong Cheng, Rotorua (NZ)

Correspondence Address: KNOBBE MARTENS OLSON & BEAR LLP, 2040 MAIN STREET, FOURTEENTH FLOOR, IRVINE, CA, 92614, US

(21) Appl. No.: 12/148,333

(22) Filed: Apr. 18, 2008

Related U.S. Application Data
(60) Provisional application No. 60/925,165, filed on Apr. 18, 2007.

(51) Int. Cl. A01H 5/00

(52) U.S. Cl. PLT/156

(57) ABSTRACT

A new and distinct kiwifruit variety is disclosed. The variety results from selection among a population of seedlings derived from crossing the kiwifruit selections known as CK51_06 (not patented) and CK51_11 (not patented). The fruit of the plant of this new kiwifruit variety is characterized by its internal red color and early harvest maturity. The new kiwifruit variety has been named ‘Hort22D’.

### LATIN NAME OF THE GENUS AND SPECIES OF THE PLANT CLAIMED

Actinidia chinensis

### VARIETY DENOMINATION ‘HORT22D’

### CROSS-REFERENCE TO RELATED APPLICATIONS

This application claims the benefit of U.S. Provisional Application No. 60/925,165, filed on Apr. 18, 2007, the entire contents of which are hereby incorporated by reference and should be considered a part of this specification.

### BACKGROUND AND SUMMARY OF THE INVENTION

The invention relates to the discovery and asexual propagation of a new and distinct variety of kiwifruit, Actinidia chinensis cv. ‘Hort22D’, as herein described and illustrated. The new kiwifruit variety ‘Hort22D’ was selected from a population of seedlings derived from crossing two unpatented kiwifruit selections CK51_{—}06 (unpatented) and CK51_{—}11 (unpatented) in the course of a planned kiwifruit variety breeding program. The cross was made in October 1996 by the Horticulture and Food Research Institute of New Zealand Limited at Te Puke, Bay of Plenty, New Zealand. The new variety was selected as 52.13.22D (breeder reference) and has been named ‘Hort22D’. The new kiwifruit variety ‘Hort22D’ may be distinguished from presently available Actinidia chinensis cultivars by the following, distinguishing characteristics. The fruit of the new kiwifruit variety ‘Hort22D’ matures earlier than the fruit of ‘Hort16A’ (U.S. Plant Pat. No. 11,066). The new kiwifruit variety ‘Hort22D’ produces much sweeter fruit having a strong expression of red color in the flesh.

The new kiwifruit variety ‘Hort22D’ may be distinguished from other presently available kiwifruit varieties featuring red-flesh fruit such as, for example, ‘Hongyang’ (not patented) and ‘Red Princess’ (not patented), by the following distinguishing characteristics. The fruit shape of the new variety ‘Hort22D’ is oblong compared with that of ‘Hongyang’, which is obovoid. The fruit shape at the stylar ends of the new variety ‘Hort22D’ is slightly protruding compared with that of ‘Hongyang’, which is hollow. The fruit of ‘Hort22D’ also matures earlier and is larger in size than those of ‘Hongyang’. Furthermore, the expression of red color in flesh of the new variety ‘Hort22D’ is very strong compared with that of ‘Red Princess’, which is weak. In addition, ‘Hort22D’ is diploid compared with ‘Red Princess’, which is tetraploid.

Asexual propagation of the new kiwifruit variety ‘Hort22D’ by grafting shows that the unique combination of characteristics of the variety come true to form and are established and transmitted through succeeding propagation. In order to obtain true-to-type clones of the initial plant, asexually propagated plants were obtained by grafting dormant buds from the original seedling onto rootstocks.

### BRIEF DESCRIPTION OF THE FIGURES

The accompanying figures illustrate in full color typical specimens of the fruit, flowers, and leaves of the new variety cv. ‘Hort22D’. The colors as depicted are as nearly true as is reasonably possible in a color representation of this type.

FIG. 1 is a photograph of the fruit of the new kiwifruit variety ‘Hort22D’.

FIG. 2 is a photograph of the stem and stylar end views of the fruit of the variety ‘Hort22D’.

FIG. 3 is a photograph of the fruit of the new kiwifruit variety ‘Hort22D’ in longitudinal-section revealing the fruit's red pigmentation at maturity for consumption.

FIG. 4 is a photograph of the flowers of the new kiwifruit variety ‘Hort22D’.

FIG. 5 is a photograph of a mature leaf of the new kiwifruit variety ‘Hort22D’.

FIG. 6 is a photograph of immature leaves of the new kiwifruit variety ‘Hort22D’.

FIG. 7 is a photograph of the ovary and fruit in cross section and longitudinal section at different stages of development as determined by days after full bloom (DAFB) of the new kiwifruit variety ‘Hort22D’.

FIG. 8 is a photograph of the core cavity present in the fruit of the new kiwifruit variety ‘Hort22D’.

FIG. 9 is a photograph of the flowers of the new kiwifruit variety ‘Hort22D’ on a vine.

FIG. 10 is a photograph of the fruit of the new kiwifruit variety ‘Hort22D’ on a vine.

### DETAILED BOTANICAL DESCRIPTION OF THE INVENTION

The following is a detailed description of the new variety. The specimens described were grown at Te Puke, Bay of Plenty, New Zealand. The observations were made in the 2003 to 2007 seasons on vines grafted onto existing clonal ‘Bruno’ rootstock, and managed under standard orchard practice. Random measurements of each characteristic were obtained from samples of 10 plants unless otherwise stated.

Horticultural terminology is used in accordance with UPOV guidelines for kiwi. All dimensions are in millimeters, and all weights are in grams (unless otherwise stated). Certain characteristics of this variety, such as growth and color, may change with changing environmental conditions (e.g., light, temperature, moisture), nutrient availability, rootstocks, or other factors. Color descriptions and other terminology are used in accordance with their ordinary dictionary descriptions, unless, the context clearly indicates otherwise. Color names beginning with a capital letter designate values based upon the R.H.S. Color Chart published by The Royal Horticultural Society, London, England.

### PLANT AND FOLIAGE

The plant is a female plant that is diploid and expresses a twining habit of low to medium vigor.

Tomentose hairs are present on the young shoot at medium density. Anthocyanin (Red) coloration of the growing tip is present (RHS 181B) (FIG. 6).

The stem of the plant is smooth bark and colored Dark-brown (RHS 200B) on the upper side. Hairs are absent from the plant stem, but there are a few White lenticels (RHS 159C). The size of the bud support is small and the leaf scar is shallow. Stems are of medium thickness, averaging 12.4 mm in diameter and buds on these stems are 7.6 mm wide on average. A bud cover is present on these stems.

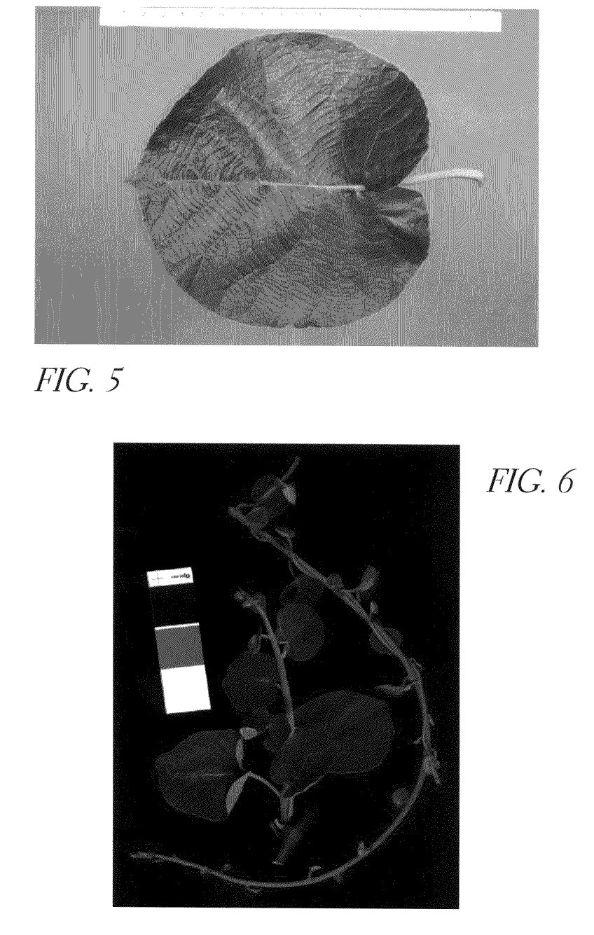

The mature leaf is broad ovate in shape with a rounded leaf tip (FIG. 5) and typically averages approximately 132 mm in length and approximately 140 mm in width. The leaf bases are slightly overlapping. The color of the leaf blade is Medium Dark-green (RHS 139A) on the upper surface and Yellow-green (RHS 137C) on the lower surface. Leaves have a few hairs, but only on the mid rib of the leaf surface. Leaf petioles average approximately 124 mm and have medium anthocyanin coloration on the upper side of the petiole in some cases (RHS 58C), but petioles are predominantly a Grey-yellow color (RHS 160A). Petioles have a relatively dense covering of hairs.

### INFLORESCENCE

The number of flowers in each inflorescence is typically between one to four flowers, with each inflorescence comprising a single king flower and one or two side flowers (0.4 per inflorescence on average). Each one-year-old shoot, typically develops three or four inflorescences, with an average of 3.6.

Pedicels average approximately 38.1 mm in length and are densely covered in short hair.

There are between six and eight petals on each flower, with an average of 6.5. The petals are arranged overlapping and are White in color (RHS 158C) when fully open, but with a Light-green base (RHS 144B) (FIG. 4). Petals are weakly curved at the apex. Flowers also contain between five and six sepals that are Green in color (RHS 145B). Each flower produces between 31 and 48 styles (39 on average) with a semi-erect attitude, that are six mm long on average and White in color (RHS 155D). Between 50 to 68 stamens (55 on average) are also present on each flower, with filaments that are seven mm long on average and vary from Green in color at their base (RHS 144B) to White in color near the anthers (RHS 155D). Anthers are three mm long on average and are Yellow-orange in color (RHS 18A).

Ovaries of the king flower are covered in fine hairs that are White in color (RHS 155D). Anthocyanin (Red) coloration of the ovary in cross section is present in the outer pericarp (RHS 50B). Ovaries of the king flower have a mean length of six mm and are oblong in shape.

### FRUIT

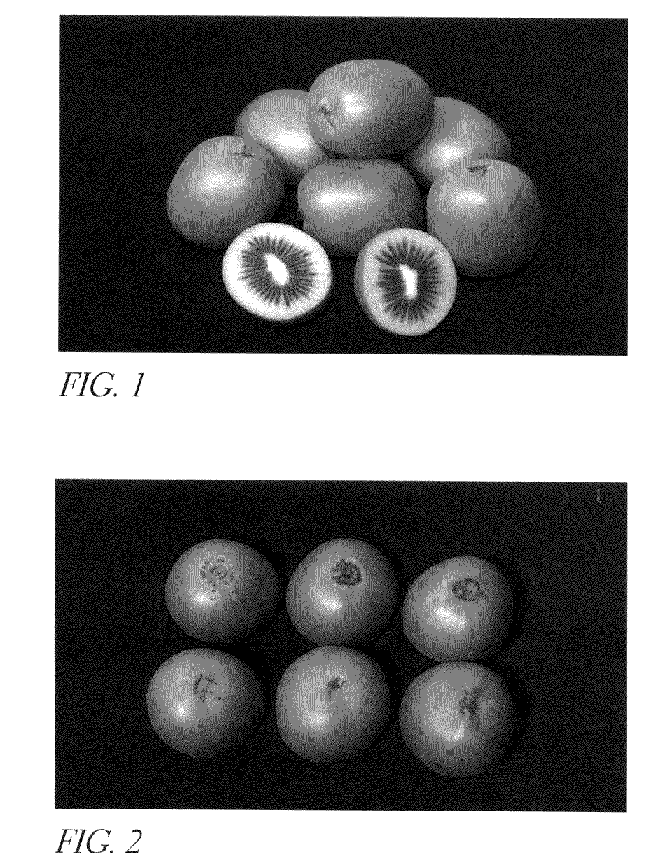

The fruit overall are small to medium in size, averaging approximately 69.2 g in weight. The dimensions of the fruit average 51.6 mm in length and 49.0 mm maximum width, with maximum core diameter averaging 18.4 mm. The general fruit shape in longitudinal section is oblong, with the general shape of the stylar end being slightly blunt protruding (FIGS. 1 and 2). In cross section, the fruit shape at the median part of the fruit varies between circular and elliptic, with most fruit being at least partially elliptic in shape.

Fruit stalks are short, averaging 38 mm long and 2.8 mm in diameter and are Brown in color (RHS 199B).

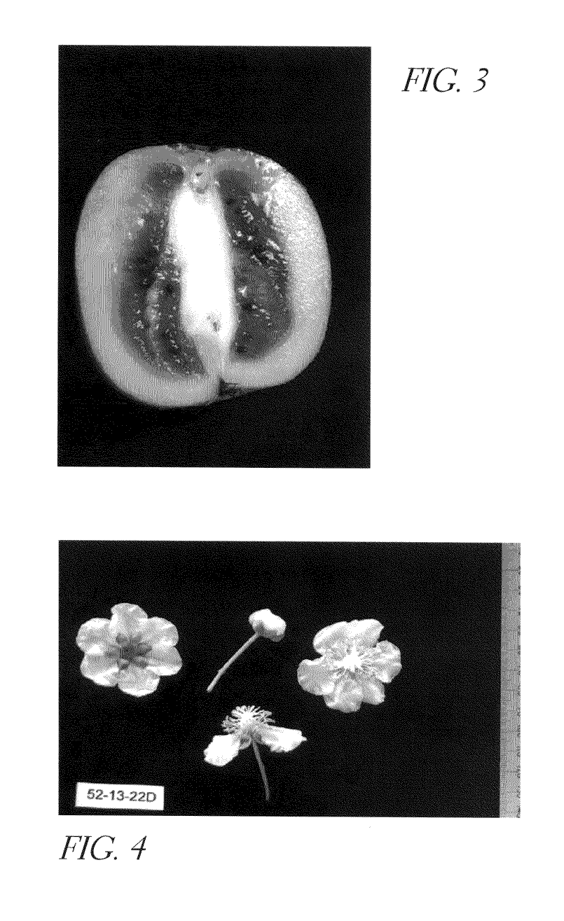

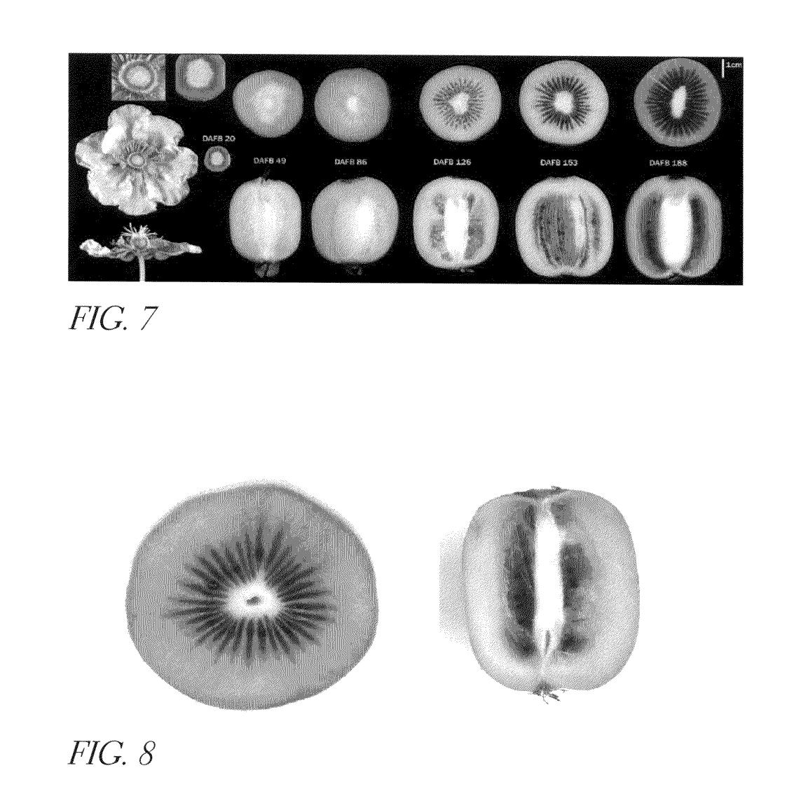

Downy, medium density fruit hairs are present on the young fruitlets, but hairs are very short and the fruit appear smooth and almost hairless when mature. The skin of the fruit remains Green in color (RHS 146B) when the fruit have been completely protected from direct sun exposure, but the skin changes to Brown in color (RHS 165B) on exposed fruit. The skins are covered with numerous lenticels that are Light Orange-white in color (RHS 159C), but are relatively inconspicuous. The outer pericarp loses the anthocyanin pigmentation within 35 days of flowering and becomes a Light-green color (RHS 149B). The outer pericarp then gradually changes to a Yellow color (RHS 11B) over a six to eight week period, as the fruit mature during March and early April in the Te Puke region of New Zealand. The inner pericarp is colored Red (RHS 45B) at maturity for consumption (FIG. 3), with anthocyanin pigments evident in the inner pericarp from December in the Te Puke region of New Zealand (FIG. 7). The core of the mature fruit is White (RHS 158D) and irregularly elliptic in shape. In almost all (93%) of the fruit, a cavity can be observed near the base of the core (FIG. 8). The fruit contain between 32 and 41 locules (37 on average) and between 20 and 36 seeds (27 on average) can be counted on a transverse slice made through the middle of the fruit. Mature seeds are Dark-brown in color (RHS 200A).

Fruit flavor is very sweet with flavor characteristics of both ‘Hayward’ and ‘Hort16A’ kiwifruit. The fruit start to ripen soon after they have been harvested in late March and early April in the Te Puke region of New Zealand and will reach eating ripeness within two weeks if they are not stored at low temperatures. Fruit that are harvested before they are ripe will continue to accumulate soluble solids as starch is converted to soluble sugars. The fruit average 16.8% soluble solids (measured as Brix) and contain 198 mg of vitamin C per 100 g of fresh weight at maturity for consumption.

### CULTIVATION

‘Hort22D’ plants can be grown on the same rootstocks as other standard varieties such as ‘Hayward’ and ‘Hort16A’. Rootstocks currently being used in New Zealand include A. deliciosa seedlings, A. chinenis seedlings, and ‘Kaimai’ (not patented).

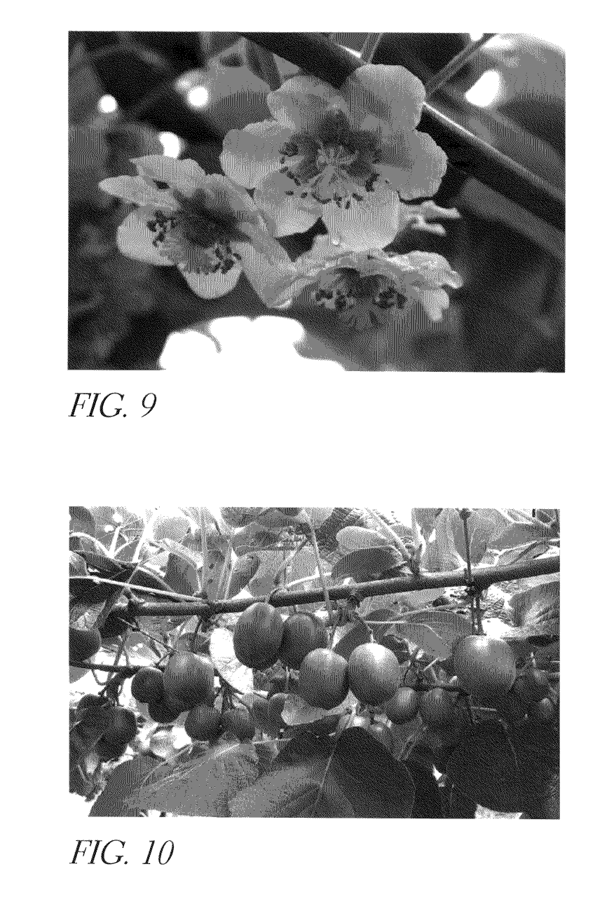

Vegetative bud break typically occurs in late August in the Te Puke region of New Zealand, with flowering commencing in mid October (FIG. 9). Between 43% and 68% of dormant buds burst in spring. The fruit attain maturity for harvest when they reach 10° Brix and 103° hue angle of the outer pericarp in approximately late March to early April in the Te Puke region of New Zealand. However, the harvest of fruit for long term cold storage is possible as early as the start of March in the Te Puke region of New Zealand. Leaves persist on the plants until early winter (June to July) in the Te Puke region of New Zealand.

Plants produce very high flower numbers, with between 50 and 140 flowers per two-metre-long cane and crop loads of between 100 and 150 fruit per square meter of canopy produced in the absence of bud-break enhancing agents in the Te Puke region of New Zealand, prior to fruit thinning (FIG. 10). Fruit yield is largely determined by the proportion of fruit that can be produced without surface markings and the proportion of fruit that have an acceptable fruit shape. To obtain a high proportion of marketable yield, ‘Hort22D’ fruit must be well protected from wind and direct sun for the first 90 days after flowering. Heavy thinning is required to reduce fruit numbers to a manageable crop load, to remove marked fruit and to remove fruit that do not meet grade standards for fruit shape. Natural fruit drop occurs prior to harvest, with 21% of fruit initially set dropping naturally from the plants during March in 2006 in the Te Puke region of New Zealand.

In post harvest storage, softening of harvested fruit can be delayed by placing fruit into cold storage in normal atmosphere or with a controlled atmosphere. In this case, fruit can be successfully stored for between 8 and 12 weeks while maintaining average flesh firmness greater than one kgf. Once removed from long term cold store and returned to ambient temperatures, the fruit continue to soften and must be consumed within approximately four days.

An additional surface marking that appears as dark spots on the skin sometimes develops between January and March and can be partially controlled with fungicide applications. Fruit are susceptible to Sclerotinia fungal infections soon after flowering. Results of controlled inoculations suggest that plants are substantially resistant to Latania Scale (Hemiberlesia lataniae) insects.

We claim:

1. A new and distinct kiwifruit variety substantially as herein described and illustrated, characterized by fruit with strong internal red color, oblong shape, and early harvest maturity.
